(19) United States
(12) Patent Application Publication
(10) Pub. No.: US 2016/0228569 A1
(43) Pub. Date: Aug. 11, 2016

(54) PEPTIDE CONSTRUCTS AND WELL-DEFINED AGGREGATES THEREOF

(71) Applicant: S. Kenny ROBERTS, Phoenixville, PA (US)

(72) Inventor: S. Kenny Roberts, Phoenixville, PA (US)

(21) Appl. No.: 15/029,487

(22) PCT Filed: Oct. 15, 2014
(86) PCT No.: PCT/US14/60662

§ 371 Date: Apr. 14, 2016

Related U.S. Application Data
(60) Provisional application No. 61/891,014, filed on Oct. 15, 2013.

(51) Int. Cl. A61K 47/48; C07K 5/103

(52) U.S. Cl. CPC A61K 47/48246; C07K 5/101; A61K 47/48338; A61K 47/48876; A61K 47/48884; C07K 2319/00

(57) ABSTRACT

This invention relates to isolated peptides, peptide mimetics and repeat units thereof, which are capable of self-assembly into nano-microparticles or microparticles, which are useful for a range of application including drug delivery.

This application claims the benefit of priority of U.S. Provisional Application No. 61/891,014, filed Oct. 15, 2013, which is incorporated herein by reference in its entirety.

### TECHNICAL FIELD

This invention relates to libraries of isolated aggregating peptides, peptide mimetics and repeat units thereof, defined aggregates, nanostructures, microstructures, nanoparticles, and applications thereof.

### BACKGROUND OF THE INVENTION

Many potent and highly specific drugs have yet to reach patients due to their rapid rate of clearance from circulation. There is a growing need for new material to fulfill unmet needs in basic research but also in the pharmaceutical industry to help get the best drugs to the patients. As a result, researchers and the pharmaceutical industry are increasingly turning their attention to peptide-based materials both as therapeutics and nanotherapeutics. Gebauer, M. & Skerra, A. Engineered protein scaffolds as next-generation antibody therapeutics. Current opinion in chemical biology 13, 245-255, doi:10.1016/j.cbpa.2009.04.627 (2009); Skerra, A. Alternative non-antibody scaffolds for molecular recognition. Current opinion in biotechnology 18, 295-304, doi:10.1016/j.copbio.2007.04.010 (2007); McGonigle, P. Peptide therapeutics for CNS indications. Biochemical pharmacology 83, 559-566, doi:10.1016/j.bcp.2011.10.014 (2012).

Peptide-based nanoparticles devices (PBND) are one interesting class of new material and have been shown to form nanostructures, including nanoparticles capable of delivering peptides, protein, and siRNA. These constructs can not only be used as passive drug carriers and in 2D and 3D cell culture systems, but also have potential to target and bind directly to cells as well as being incorporated into the extracellular matrix. S Zhang, X. Z., L Spirio. in Scaffolding in Tissue Engineering (ed Ma and Elisseeff) 217-238 (CRC Press, 2005); Branco, M. C., Sigano, D. M. & Schneider, J. P. Materials from peptide assembly: towards the treatment of cancer and transmittable disease. Current opinion in chemical biology 15, 427-434, doi:10.1016/j.cbpa.2011.03.021 (2011).

### SUMMARY OF THE INVENTION

The invention described herein relates to libraries of isolated aggregating peptidesisolated peptides, peptide mimetic and sequential or random repeat units that can be used to extend the half-life of drug, improve drug stability, and specificity. The invention also describes methods for engineering and synthesis, certain aspects of composition, articles, and kits comprising the isolated peptide constructs.

Accordingly, one aspect of the invention provides an isolated peptide or an aggregated peptide construct consisting essentially of chemically linked amino acid residues of the following sequence (X₁—X₂—X₃—X₄) wherein X₁ is isoleucine (Ile) or a conservative substitution thereof; X₂ is proline (Pro) or a conservative substitution thereof; X₃ is glycine (Gly) or a conservative substitution thereof; X₄ is tyrosine (Tyr) or a conservative substitution thereof. The invention also describes an isolated peptide, wherein X₃ is independently selected from a bond, a non-coded, non-proteinogenic or non-standard amino acid linker including but not limited to β-alanine, γ-Aminobutyric acid (GABA), δ-Aminolevulinic acid, aminoisobutyric acid, dehydroalanine, PEG.

Accordingly, one embodiment of this invention includes an isolated peptide or an aggregated peptide construct comprising chemically linked amino acid residues of the following sequence: X₁—X₂—X₃—X₄; where X₁ is an L- or D-amino acid; X₂ is proline, or a conservative substitution thereof; X₃ is selected from a group consisting of glycine or a conservative substitution thereof, a bond, and a non-coded, non-proteinogenic, or a non-standard amino acid linker; X₄ is an L- or D-amino acid; and said peptide is terminated at the amino- and/or carboxy-terminus with a biological entity.

A further embodiment of this invention includes the isolated peptide, where X₁ is isoleucine or a conservative substitution thereof. A further embodiment of this invention also includes the isolated peptide, where X₁ is threonine or a conservative substitution thereof. A further embodiment of this invention also includes the isolated peptide, where X₁ is lysine or a conservative substitution thereof.

Another further embodiment of this invention includes the isolated peptide or construct, where said non-coded, non-proteinogenic, or non-standard amino acid linker is selected from the group consisting of β-alanine, γ-Aminobutyric acid (GABA), δ-Aminolevulinic acid, aminoisobutyric acid, dehydroalanine, and PEG.

Another further embodiment of this invention includes the isolated peptide or construct, where said biological entity is selected from a group consisting of an amine, amide, imine, imide, azide, azo compound, carboxylic acid, carbonate, carboxylate, ester, alcohol, aldehyde, alkane, alkene, alkyne, halogens, ketone, acyl halide, boronic acid, boronic ester, borinic acid, borinic ester, hydroperoxide, peroxide, ether, hemiacetal, hemiketal, acetal, ketal, orthoester, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, fatty acid, myristic acid, palmytolyl, Fluorenylmethyloxycarbonyl or FMOC carbamate, Z (CBZ), Boc, tert-Butyl, cell surface receptor ligand, an antibody, bispecific antibody, an antibody-like molecule, Fab, Fc, or a portion thereof, an aptamer, a cytokine, hormone, a lectin, a lipid, nucleic acid, a carbohydrate, enzyme, biotin, avidin, streptavidin, steroid, protein A, protein G, a plasma albumin, a ligand, a therapeutic agent, fluorescent molecule, a binding molecule, a biodegradable non-amino acid polymer and any combinations thereof.

In some embodiments, the following sequence (X₁—X₂—X₃—X₄) is repeated sequentially or randomly to form polypeptides of the following length: 11, 13, 17, 19, 22, 23, 26, 28, 29, 31, 33, 34, 36, 37, 38, 39, 41, 43, 44, 46, 47, 51, 52, 53, 57, 58, 59.

In some embodiments, the isolated peptide comprises a chemically linked amino acid residue having the sequence X₁—X₂—X₃—X₄.

In some embodiments, the isolated peptide consists of a chemically linked amino acid residue has the sequence H—X₁—X₂—X₃—X₄—OH. When a H is shown, it is part of the amino group of the amino acid residue, while OH is part of the carboxyl group of the amino acid residue.

In some embodiments, the isolated peptide consists of a chemically linked amino acid residue has the sequence HO—X₁—X₂—X₃—X₄—H.

In some embodiments of the isolated peptide comprises a chemically linked amino acid residue having the sequence X₁—X₂—X₃—X₄; the isolated peptide consisting of a chemically linked amino acid residue having the sequence X₁—X₂—X₃—X₄; the isolated peptide consisting of a chemically linked amino acid residue having the sequence H—X₁—X₂—X₃—X₄—OH; or the isolated peptide consisting of a chemically linked amino acid residue having the sequence HO—X₁—X₂—X₃—X₄—H: X₁ is isoleucine (IIe), glutamic acid (Glu), tyrosine (Tyr), valine (Val), or lysine (Lys). In some embodiments, X₁ is isoleucine (IIe). In some embodiments, X₁ is glutamic acid (Glu). In some embodiments, X₁ is tyrosine (Tyr). In some embodiments, X₁ is valine (Val). In some embodiments, X₁ is lysine (Lys). In some embodiments, X₁ is isoleucine or a conservative substitution thereof. In some embodiments, X₂ is proline. In some embodiments, X₃ is glycine. In some embodiments, X₄ is isoleucine (IIe), tyrosine (Tyr), histidine (His), or phenylalanine (Phe). In some embodiments, X₄ is isoleucine (IIe). In some embodiments, X₄ is tyrosine (Tyr). In some embodiments, X₄ is histidine (His). In some embodiments, X₄ is phenylalanine (Phe). In some embodiments, X₁ is isoleucine (IIe), glutamic acid (Glu), tyrosine (Tyr), valine (Val), or lysine (Lys); X₂ is proline; X₃ is glycine; and X₄ is isoleucine (IIe), tyrosine (Tyr), histidine (His), or phenylalanine (Phe). In some embodiments, X₁ is isoleucine (IIe), X₂ is proline, X₃ is glycine, and X₄ is isoleucine (IIe). In some embodiments, X₁ is glutamic acid (Glu), X₂ is proline, X₃ is glycine, and X₄ is isoleucine (IIe). In some embodiments, X₁ is tyrosine (Tyr), X₂ is proline, X₃ is glycine, and X₄ is isoleucine (IIe). In some embodiments, X₁ is valine (Val), X₂ is proline, X₃ is glycine, and X₄ is isoleucine (IIe). In some embodiments, X₁ is lysine (Lys), X₂ is proline, X₃ is glycine, and X₄ is isoleucine (IIe). In some embodiments, X₁ is isoleucine (IIe), X₂ is proline, X₃ is glycine, and X₄ is tyrosine (Tyr). In some embodiments, X₁ is glutamic acid (Glu), X₂ is proline, X₃ is glycine, and X₄ is tyrosine (Tyr). In some embodiments, X₁ is tyrosine (Tyr), X₂ is proline, X₃ is glycine, and X₄ is tyrosine (Tyr). In some embodiments, X₁ is valine (Val), X₂ is proline, X₃ is glycine, and X₄ is tyrosine (Tyr). In some embodiments, X₁ is lysine (Lys), X₂ is proline, X₃ is glycine, and X₄ is tyrosine (Tyr). In some embodiments, X₁ is isoleucine (IIe), X₂ is proline, X₃ is glycine, and X₄ is histidine (His). In some embodiments, X₁ is glutamic acid (Glu), X₂ is proline, X₃ is glycine, and X₄ is histidine (His). In some embodiments, X₁ is tyrosine (Tyr), X₂ is proline, X₃ is glycine, and X₄ is histidine (His). In some embodiments, X₁ is valine (Val), X₂ is proline, X₃ is glycine, and X₄ is histidine (His). In some embodiments, X₁ is lysine (Lys), X₂ is proline, X₃ is glycine, and X₄ is histidine (His). In some embodiments, X₁ is isoleucine (IIe), X₂ is proline, X₃ is glycine, and X₄ is phenylalanine (Phe). In some embodiments, X₁ is glutamic acid (Glu), X₂ is proline, X₃ is glycine, and X₄ is phenylalanine (Phe). In some embodiments, X₁ is tyrosine (Tyr), X₂ is proline, X₃ is glycine, and X₄ is phenylalanine (Phe). In some embodiments, X₁ is valine (Val), X₂ is proline, X₃ is glycine, and X₄ is phenylalanine (Phe). In some embodiments, X₁ is lysine (Lys), X₂ is proline, X₃ is glycine, and X₄ is phenylalanine (Phe).

In some embodiments, the isolated peptide is terminated with an 8-15 amino acid sequence, wherein the amino acids are selected from cysteine (C), glycine (Gly), histidine (His), arginine (Arg), serine (Ser), and phenylalanine (Phe). In some embodiments, the 8-15 amino acid sequence comprises a sequence -Cys-His-His-His-Arg-His-Ser-Phe.

In some embodiments, wherein the isolated peptide is selected from:

H-Val-Pro-Gly-Tyr-OH;
H-Val-Pro-Gly-Phe-OH;
H-Val-Pro-Gly-Ile-OH;
H-Val-Pro-Gly-His-OH;
H-Val-Pro-Gly-Trp-OH;
H-Lys-Pro-Gly-Tyr-OH;
H-Glu Pro-Gly-Tyr-OH;
H-Lys-Pro-Gly-Phe-OH;
H-Glu-Pro-Gly-Phe-OH;
H-Ile-Pro-Gly-Tyr-OH;
H-Thr-Pro-Gly-Tyr-OH;
H-Ile-Pro-Gly-Phe-OH;
and
H-Thr-Pro-Gly-Phe-OH.

In some embodiments, the isolated peptide is selected from:

VPGY-CHHHRHSF;
VPGY-G-CHHHRHSF;
VPGY-GS-CHHHRHSF;
and
VPGY-GGGS-CHHHRHSF.

Another embodiment of this invention includes an isolated peptide or an aggregated peptide construct comprising chemically linked amino acid residues of the following sequence: (X₅—X₆—X₇—X₈)ₙ; where X₅ is selected from one of the 21 naturally occurring amino acids; X₆ is proline, or a conservative substitution thereof; X₇ is selected from a group consisting of glycine or a conservative substitution thereof, a bond, and a non-coded, non-proteinogenic, or a non-standard amino acid linker; X₈ is selected from one of the 21 naturally occurring amino acids; N is an integer from 1-50, inclusive; and said peptide is terminated with a biological entity.

Another embodiment includes an aggregated peptide construct comprising an isolated peptide comprising chemically linked amino acid residues selected from the group of the following sequences X₁—X₂—X₃—X₄; and (X₁—X₂—X₃—X₄)ₙ.

In some embodiments, the isolated peptide comprising chemically linked amino acid residues X₁—X₂—X₃—X₄ does not have an additional glycine residue bonded directly to X₄; and the isolated peptide comprising chemically linked amino acid residues (X₅—X₆—X₇—X₈)ₙ does not have an additional glycine residue bonded directly to X₈.

In some embodiments, the isolated peptide comprising chemically linked amino acid residues X₁—X₂—X₃—X₄ does not have an additional glycine residue bonded directly to X₄, except as part of a linking group to a terminal covalently bound therapeutic agent; and the isolated peptide comprising chemically linked amino acid residues (X₅—X₆—X₇—X₈)ₙ does not have an additional glycine residue bonded directly to X₈, except as part of a linking group to a terminal covalently bound therapeutic agent.

Another embodiment includes an aggregated peptide construct consisting of an isolated peptide consisting of chemically linked amino acid residues selected from the group of the following sequences X₁—X₂—X₃—X₄; and (X₁—X₂—X₃—X₄)ₙ.

A further embodiment of this invention includes the isolated peptide, where X₅ is isoleucine or a conservative substitution thereof.

In some embodiments, the isolated peptide comprises a chemically linked amino acid residue having the sequence (X₅—X₆—X₇—X₈)ₙ.

In some embodiments, the isolated peptide consists of a chemically linked amino acid residue having the sequence H—(X₅—X₆—X₇—X₈)ₙ—OH, wherein n is 2-50 inclusive.

In some embodiments, the isolated peptide consists of a chemically linked amino acid residue having the sequence HO—(X₅—X₆—X₇—X₈)ₙ—H, wherein n is 2-50 inclusive.

In some embodiments of the isolated peptide comprises a chemically linked amino acid residue having the sequence (X₅—X₆—X₇—X₈)ₙ; the isolated peptide consisting of a chemically linked amino acid residue having the sequence (X₅—X₆—X₇—X₈)ₙ; the isolated peptide consists of a chemically linked amino acid residue having the sequence H—(X₅—X₆—X₇—X₈)ₙ—OH, wherein n is 2-50 inclusive; or the isolated peptide consists of a chemically linked amino acid residue having the sequence HO—(X₅—X₆—X₇—X₈)ₙ—H, wherein n is 2-50 inclusive, X₅ is isoleucine (IIe), glutamic acid (Glu), tyrosine (Tyr), valine (Val), or lysine (Lys). In some embodiments, X₅ is isoleucine (IIe). In some embodiments, X₅ is glutamic acid (Glu). In some embodiments, X₅ is tyrosine (Tyr). In some embodiments, X₅ is valine (Val). In some embodiments, X₅ is lysine (Lys). In some embodiments, X₆ is proline. In some embodiments, X₇ is glycine. In some embodiments, X₈ is isoleucine (Be), tyrosine (Tyr), histidine (His), or phenylalanine (Phe). In some embodiments, X₈ is isoleucine (Be). In some embodiments, X₈ is tyrosine (Tyr). In some embodiments, X₈ is histidine (His). In some embodiments, X₈ is phenylalanine (Phe). In some embodiments, X₅ is isoleucine (Be), glutamic acid (Glu), tyrosine (Tyr), valine (Val), or lysine (Lys); X₆ is proline; X₇ is glycine; and X₈ is isoleucine (Be), tyrosine (Tyr), histidine (His), or phenylalanine (Phe). In some embodiments, X₅ is isoleucine (Be), X₆ is proline, X₇ is glycine, and X₈ is isoleucine (IIe). In some embodiments, X₅ is glutamic acid (Glu), X₆ is proline, X₇ is glycine, and X₈ is isoleucine (Be). In some embodiments, X₅ is tyrosine (Tyr), X₆ is proline, X₇ is glycine, and X₈ is isoleucine (IIe). In some embodiments, X₅ is valine (Val), X₆ is proline, X₇ is glycine, and X₈ is isoleucine (IIe). In some embodiments, X₅ is lysine (Lys), X₆ is proline, X₇ is glycine, and X₈ is isoleucine (IIe). In some embodiments, X₅ is isoleucine (Be), X₆ is proline, X₇ is glycine, and X₈ is tyrosine (Tyr). In some embodiments, X₅ is glutamic acid (Glu), X₆ is proline, X₇ is glycine, and X₈ is tyrosine (Tyr). In some embodiments, X₅ is tyrosine (Tyr), X₆ is proline, X₇ is glycine, and X₈ is tyrosine (Tyr). In some embodiments, X₅ is valine (Val), X₆ is proline, X₇ is glycine, and X₈ is tyrosine (Tyr). In some embodiments, X₅ is lysine (Lys), X₆ is proline, X₇ is glycine, and X₈ is tyrosine (Tyr). In some embodiments, X₅ is isoleucine (IIe), X₆ is proline, X₇ is glycine, and X₈ is histidine (His). In some embodiments, X₅ is glutamic acid (Glu), X₆ is proline, X₇ is glycine, and X₈ is histidine (His). In some embodiments, X₅ is tyrosine (Tyr), X₆ is proline, X₇ is glycine, and X₈ is histidine (His). In some embodiments, X₅ is valine (Val), X₆ is proline, X₇ is glycine, and X₈ is histidine (His). In some embodiments, X₅ is lysine (Lys), X₆ is proline, X₇ is glycine, and X₈ is histidine (His). In some embodiments, X₅ is isoleucine (IIe), X₆ is proline, X₇ is glycine, and X₈ is phenylalanine (Phe). In some embodiments, X₅ is glutamic acid (Glu), X₆ is proline, X₇ is glycine, and X₈ is phenylalanine (Phe). In some embodiments, X₅ is tyrosine (Tyr), X₆ is proline, X₇ is glycine, and X₈ is phenylalanine (Phe). In some embodiments, X₅ is valine (Val), X₆ is proline, X₇ is glycine, and X₈ is phenylalanine (Phe). In some embodiments, X₅ is lysine (Lys), X₆ is proline, X₇ is glycine, and X₈ is phenylalanine (Phe).

In some embodiments, n is an integer from 1 to 2.

In some embodiments, n is 1 and said isolated peptide is terminated with a 4-7 amino acid sequence, wherein the amino acids are selected from proline (Pro), glycine (Gly), isoleucine (IIe), glutamic acid (Glu), tyrosine (Tyr), valine (Val), lysine (Lys), histidine (His), and phenylalanine (Phe), wherein at least one amino acid is Pro and at least one amino acid is Gly.

Another further embodiment of this invention includes the isolated peptide, where the non-coded, non-proteinogenic, or non-standard amino acid linker is selected from the group consisting of β-alanine, γ-Aminobutyric acid (GABA), δ-Aminolevulinic acid, aminoisobutyric acid, dehydroalanine, and PEG.

Another further embodiment of this invention includes the isolated peptide, where the biological entity is selected from a group consisting of an amine, amide, imine, imide, azide, azo compound, carboxylic acid, carbonate, carboxylate, ester, alcohol, aldehyde, alkane, alkene, alkyne, halogens, ketone, acyl halide, boronic acid, boronic ester, borinic acid, borinic ester, hydroperoxide, peroxide, ether, hemiacetal, hemiketal, acetal, ketal, orthoester, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, fatty acid, myristic acid, palmytolyl, Fluorenylmethyloxycarbonyl or FMOC carbamate, Z (CBZ), Boc, tert-Butyl, cell surface receptor ligand, an antibody, bispecific antibody, an antibody-like molecule, Fab, Fc, or a portion thereof, an aptamer, a cytokine, hormone, a lectin, a lipid, nucleic acid, a carbohydrate, enzyme, biotin, avidin, streptavidin, steroid, protein A, protein G, a plasma albumin, a ligand, a therapeutic agent, fluorescent molecule, a binding molecule, a biodegradable non-amino acid polymer and any combinations thereof. In some embodiments, the biological entity is selected from DNA, siRNA, and mRNA.

In some embodiments, the isolated peptide has a terminal covalently linked therapeutic agent. In some embodiments, the isolated peptide has a terminal covalently linked therapeutic agent, wherein said therapeutic agent is covalently linked to the peptide via a linker. In some embodiments, the linker is selected from GS, (GS)ₘ GGGS, and (GGGS)m, wherein m is 1-25. In some embodiments, m is 1. In some embodiments, the linker is one described at http://parts.igem.org/Protein_domains/Linker, which is incorporated herein by reference in its entirety.

In some embodiments, the isolated peptide is selected from:

H-Val-Pro-Gly-Tyr-Val-Pro-Gly-Tyr-Val-Pro-Gly-OH;
H-Ile-Pro-Gly-Tyr-Ile-Pro-Gly-Tyr-Ile-Pro-Gly-OH;
H-Val-Pro-Gly-Tyr-Val-Pro-Gly-Tyr-Val-Pro-Lys-OH;
H-Ile-Pro-Gly-Tyr-Ile-Pro-Gly-Tyr-Ile-Pro-Lys-OH;
H-Val-Pro-Gly-Tyr-Val-Pro-Gly-Tyr-Val-Pro-His-OH;
H-Ile-Pro-Gly-Tyr-Ile-Pro-Gly-Tyr-Ile-Pro-His-OH;
H-Pro-Val-Gly-Tyr-Val-Pro-Gly-Phe-OH;
H-Val-Pro-Gly-Tyr-Pro-Val-Gly-Phe-OH;
H-Val-Phe-Pro-Gly-Tyr-Pro-Val-Gly-OH;
H-Gly-Pro-Val-Gly-Tyr-Val-Gly-Pro-Phe-Gly-OH;
H-Tyr-Gly-Val-Gly-Phe-Val-Gly-Pro-Gly-Pro-OH;
and
H-Tyr-Gly-Pro-Val-Tyr-Gly-Pro-Val-OH.

Another embodiment of this invention includes an isolated peptide or an aggregated peptide construct comprising chemically linked amino acid residues selected from the group of the following sequences: X₁₁—X₁₂—X₁₁—X₁₀—X₉, and (X₁₁—X₁₂—X₁₁—X₁₀—X₉)ₙ, wherein X₉ is an L- or D-amino acid; X₁₀ is proline, or a conservative substitution thereof; X₁₁ is selected from a group consisting of glycine or a conservative substitution thereof; X₁₂ is an L- or D-amino acid; n is an integer from 1-50, inclusive; and said peptide is terminated with chemical group, molecule, peptide blocking group, peptide, or biological entity; wherein the bond between X₁₁ and X₁₂ is formed the alpha-carboxyl group of X₁₁ and the alpha-amino group of X₁₂.

In some embodiments, the isolated peptide comprises a chemically linked amino acid residue having the sequence X₁₁—X₁₂—X₁₁—X₁₀—X₉.

In some embodiments, the isolated peptide comprises a chemically linked amino acid residue having the sequence (X₁₁—X₁₂—X₁₁—X₁₀—X₉)ₙ.

In some embodiments, the isolated peptide consists essentially of a chemically linked amino acid residue having the sequence X₁₁—X₁₂—X₁₁—X₁₀—X₉.

In some embodiments, the isolated peptide consists essentially of a chemically linked amino acid residue having the sequence (X₁₁—X₁₂—X₁₁—X₁₀—X₉)ₙ.

In some embodiments, the isolated peptide consists of a chemically linked amino acid residue having the sequence H—X₁₁—X₁₂—X₁₁—X₁₀—X₉—OH.

In some embodiments, the isolated peptide consists of a chemically linked amino acid residue having the sequence H—(X₁₁—X₁₂—X₁₁—X₁₀—X₉)ₙ—OH, wherein n is 2-50 inclusive.

In some embodiments of the isolated peptide comprising a chemically linked amino acid residue having the sequence X₁₁—X₁₂—X₁₁—X₁₀—X₉; the isolated peptide compriseing a chemically linked amino acid residue having the sequence (X₁₁—X₁₂—X₁₁—X₁₀—X₉).; the isolated peptide consisting essentially of a chemically linked amino acid residue having the sequence X₁₁—X₁₂—X₁₁—X₁₀—X₉; the isolated peptide consisting essentially of a chemically linked amino acid residue having the sequence (X₁₁—X₁₂—X₁₁—X₁₀—X₉).; the isolated peptide consisting of a chemically linked amino acid residue having the sequence H—X₁₁—X₁₂—X₁₁—X₁₀—X₉—OH; and the isolated peptide consisting of a chemically linked amino acid residue having the sequence H—(X₁₁—X₁₂—X₁₁—X₁₀—X₉)ₙ—OH, wherein n is 2-50 inclusive: X₉ is valine. In some embodiments, X₁₀ proline. In some embodiments, X₁₁ is glycine. In some embodiments, X₁₂ is tyrosine (Tyr) or phenylalanine (Phe). In some embodiments, X₁₂ is tyrosine (Tyr). In some embodiments, X₁₂ is phenylalanine (Phe). In some embodiments, X₉ is valine; X₁₀ proline; X₁₁ is glycine; and X₁₂ is tyrosine (Tyr) or phenylalanine (Phe). In some embodiments, X₉ is valine; X₁₀ proline; X₁₁ is glycine; and X₁₂ is tyrosine (Tyr). In some embodiments, X₉ is valine; X₁₀ proline; X₁₁ is glycine; and X₁₂ is phenylalanine (Phe).

In some embodiments, said non-coded, non-proteinogenic, or non-standard amino acid linker is selected from the group consisting of β-alanine, γ-Aminobutyric acid (GABA), δ-Aminolevulinic acid, aminoisobutyric acid, dehydroalanine, and PEG.

In some embodiments, said chemical group, molecule, peptide blocking group, peptide, or biological entity is selected from a group consisting of an amine, amide, imine, imide, azide, azo compound, carboxylic acid, carbonate, carboxylate, ester, alcohol, aldehyde, alkane, alkene, alkyne, halogens, ketone, acyl halide, boronic acid, boronic ester, borinic acid, borinic ester, hydroperoxide, peroxide, ether, hemiacetal, hemiketal, acetal, ketal, orthoester, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, fatty acid, myristic acid, palmytolyl, Fluorenylmethyloxycarbonyl or FMOC carbamate, Z (CBZ), Boc, tert-Butyl, cell surface receptor ligand, an antibody, bispecific antibody, an antibody-like molecule, Fab, Fc, or a portion thereof, an aptamer, a cytokine, hormone, a lectin, a lipid, nucleic acid, a carbohydrate, enzyme, biotin, avidin, streptavidin, steroid, protein A, protein G, a plasma albumin, a ligand, a therapeutic agent, fluorescent molecule, a binding molecule, a biodegradable non-amino acid polymer and any combinations thereof. In some embodiments, the biological entity is selected from DNA, siRNA, and mRNA.

In some embodiments, the isolated peptide has a terminal covalently linked therapeutic agent.

In some embodiments, the isolated peptide has a terminal covalently linked therapeutic agent, wherein said therapeutic agent is covalently linked to the peptide via a linker. In some embodiments, the linker is selected from GS, (GS)ₘ GGGS, and (GGGS)m, wherein m is 1-25. In some embodiments, m is 1. In some embodiments, the linker is one described at http://parts.igem.org/Protein_domains/Linker, which is incorporated herein by reference in its entirety.

In some embodiments, n is 2.

In some embodiments, the isolated peptide is selected from:

H-Gly-Tyr-Gly-Pro-Val-OH;
and
H-Gly-Phe-Gly-Pro-Val-Gly-Tyr-Gly-Pro-Val-OH.

Another embodiment of this invention includes the isolated peptide, where the amino acid sequence is selected from the group consisting of:

IPGY;
VPGY;
LPGY;
IPGF;
VPGF;
LPGF;
VPGW;
IPGW;
LPGW;
IPGY-VPGY-VPG;
IPGY-IPGY-IPG;
VPGY-VPGY-VPK;
IPGY-IPGY-IPK;
VPGY-VPGY-VPH;
IPGY-IPGY-IPH;
VPGY-VPGF-VPGY-V;
VPGY-VPGY-VPGY-V;
VPGY-VPGY-VPGY-L;
VPGY-VPGY-VPGY-VPGY-V;
VPGY-VPGY-VPGY-VPGY-VPG;
VPGY-VPGY-VPGY-VPGY-VPGY-VP;
VPGY-VPGY-VPGY-VPGY-VPGY-VPG;
VPGY-VPGY-VPGY-VPGY-VPGY-VPGY-VP;
VPGY-VPGY-VPGY-VPGY-VPGY-VPGY-VPGY;
VPGY-VPGY-VPGY-VPGY-VPGY-VPGY-VPGY-V;
VPGY-VPGY-VPGY-VPGY-VPGY-VPGY-VPGY-VPG;
VPGY-VPGY-VPGY-VPGY-VPGY-VPGY-VPGY-VPGY-V;
and
VPGY-VPGY-VPGY-VPGY-VPGY-VPGY-VPGY-VPGY-VP.

In another embodiment, the invention provides an aggregated peptide construct, wherein the construct is a mixture of peptides, wherein the mixture is selected from:

VPGY:KPGY;
VPGY:EPGY;
VPGY:TPGY;
VPGY:KPGY:TPGY;
VPGY:VPGY-GGGS-CHHHRHSF;
KPGY:VPGY-GGGS-CHHHRHSF;
TPGY:VPGY-GGGS-CHHHRHSF;
VPGY:VPGY-CHHHRHSF;
VPGY:VPGY-G-CHHHRHSF;
VPGY:VPGY-GS-CHHHRHSF;
and
VPGY:VPGY-GGGS-CHHHRHSF.

Another embodiment of this invention includes a half-life extension sequence comprising one or a plurality of any of the isolated peptides previously discussed.

A further embodiment of this invention includes the half-life extension sequence that also includes a therapeutic agent, drug molecule, or prodrug. Another further embodiment of this invention includes the half-life extension sequence that also includes a nanoparticle, microparticle, liposomes, lipidoids, exosomes, microvesicles, or any combination thereof. Another further embodiment of this invention includes the half-life extension sequence that also includes a quantum dot, a magnetic particle, gold nanoparticle, a silver nanoparticle, a carbon nanotube, a fullerene or any combination thereof.

Another further embodiment of this invention includes the half-life extension sequence where said therapeutic agent, drug molecule, or prodrug is selected from the group consisting of a peptide therapeutic, a protein therapeutic, a small molecule drug, siRNA, mRNA, DNA, an antibody, a bispecific antibody, a Fragment antigen binding (Fab), a F(ab′)2, Fab′, single-chain variable fragment or scFv, di-scFv, single domain antibody sd-scFv hormone, enzyme, and lipid, or any combination thereof.

Another embodiment of this invention includes a peptide aggregate comprising one or a plurality of the isolated peptides previously discussed. A further embodiment of this invention includes the aggregated peptide, where the aggregate comprises a laminar structure, a solid structure, a porous structure or a combination thereof. Another further embodiment of this invention includes the aggregated peptide, where the aggregate has a size of 3 nm to 100 microns, or 5 nm to 100 microns.

Another embodiment of this invention includes a composition comprising at least one of the isolated peptides or peptide mimetic previously discussed, a peptide aggregate, or any combination thereof, where the composition is used as an excipient in a pharmaceutical product, or in a food product.

Another embodiment of this invention includes a composition comprising at least one of the isolated peptides or peptide mimetic previously discussed and a peptide aggregate, or any combination thereof, where the composition is a personal care composition including a lotion, cream, oils, gels or shampoo, ointment or any combination thereof.

Another embodiment of this invention includes a composition comprising at least one of the isolated peptides or peptide mimetic previously discussed and a peptide aggregate, or any combination thereof, where the composition is formulated for oral administration

Another embodiment of this invention includes a method of modulating surface and material property using any of the peptides or peptide mimetics previously discussed, where said peptide or peptide mimetic is chemically crosslinked to the tissue culture surface of a tissue culture dish by any known methods in the art, using Polyethylene, Polypropylene, Polystyrene, polyvinyl chloride, or borosilicate. For example, by crosslinking the peptides to the surface using a chemical crosslinking agent, electron beam exposure, gamma-radiation, UV irradiation. Crosslinking here can also be covalent, ionic, hydrophobic or others methods know in the art.

Another embodiment of this invention includes a method for modulating a least one or more of the following biological cell behavior: cell growth, viability, apoptosis, secretion, migration, or differentiation. This method includes contacting the biological cell with the cell of at least one of the peptides or peptide mimetics previously discussed, where the peptides constructs are cross-linked to the surface of the cells using a chemical crosslinking agent, ionic, hydrophobic or other methods know in the art. For example, the peptide can be crossed linked to the cells via an amine, thiol, or carboxyl functional group.

Another embodiment of this invention includes a method of modulating the release of an active drug, drug agent, or drug substance comprising any of the peptides and/or peptide mimetics previously discussed, wherein the active drug is covalently linked, encapsulated, or embedded using ionic interactions or hydrophobic interactions in the peptide aggregates by any know method in the art, and release of the drugs occurs by degradation of the peptide, or through the natural porosity of the aggregate. The porosity can be adjusted, based on a number of factors such as, but not limited to, desired release rates, molecular size and/or diffusion coefficient of the therapeutic agent or active agent.

Another embodiment of this invention includes a kit comprising at least one container containing an isolated peptide or peptide mimetic previously discussed.

Another embodiment includes a composition comprising a construct of any one of claims 1-41 and a pharmaceutically acceptable carrier. In some embodiments, the pharmaceutically acceptable carrier is an aqueous carrier. In some embodiments, the aqueous carrier is buffered.

In some embodiments, the construct forms nanoparticles or microparticles. In some embodiments, the construct forms nanoparticles. In some embodiments, the construct forms microparticles.

In some embodiments, the composition further comprises a therapeutic agent.

In some embodiments, the composition is suitable for intravenous administration to a patient.

Another embodiment includes a method of increasing the half-life of a drug during administration to a patient, comprising administering to said patient a composition as described herein.

Another embodiment includes a method of administering a drug to a patient, comprising administering to said patient a composition as described herein.

In some embodiments, the aggregated peptide construct is self-assembling.

### BRIEF DESCRIPTION OF THE DRAWINGS

FIG. 1a and 1b show DLS size distribution plot of representative 4 amino acids peptide aggregates (nanoparticle) as a function of peptide sequence in NaCl. Each at aggregated construct formulated at 10 mg/mL (w/v) in 150 mM NaCl.

FIG. 1c-1e shows DLS size distribution plot of representative 4-11 amino acids peptide aggregates as a function of peptide sequence in NaCl (each at aggregated construct formulated at 10 mg/mL (w/v) 10 mg/mL in 150 mM NaCl).

FIG. 1f shows comparison of size distribution of representative peptide aggregates as a function of peptide sequence in NaCl and in sodium citrate (each aggregated construct formulated at 25 mg/mL (w/v) in buffer).

FIG. 2 shows size of representative peptide aggregates showing that aggregate size can be varied as a function of concentration and buffer salt concentration (cold NaCl).

FIG. 3 shows size of representative peptide aggregates showing that aggregate size can be varied as a function of peptide concentration and buffer salt concentration (cold sodium citrate).

FIG. 4a-4c show size and stability of the peptide aggregates can be controlled when formulated in buffer as a function of pH (cold sodium citrate at pH3-pH9 as compared to cold phosphate pH3-pH9). Each aggregated construct formulated at 10 mg/mL (w/v).

FIG. 5 shows DLS size distribution plot of mixtures of isolated peptide interacting to form mix aggregates in buffer. Mix aggregates formulation with either one, two or three isolated aggregating peptides mixed in 150 mM NaCl. Each aggregated mixed construct formulated at final concentration of 10 mg/mL (w/v).

FIG. 6A shows DLS size distribution plot of mixtures of isolated peptide and peptide drug fusion interacting to form mix aggregates in buffer. Mix aggregates formulated (1:1) for a final concentration of 10 mg/mL (w/v) in 15 0 mM NaCl.

FIG. 6B shows particle size as a function of citrate buffer concentration.

### DETAILED DESCRIPTION OF THE INVENTION

The invention provided herein relates to isolated peptides, peptide mimetic and their repeat units, aggregates and applications thereof.

The isolated peptides may be prepared by the formation of peptide bonds between two amino acids using know peptide synthetic strategies thus linking the amino acids together. Lloyd-Williams P. et al. (1997) Chemical approaches to the synthesis of peptides and proteins. Boca Raton: CRC Press. 278, Merrifield R. B. (1963) Solid phase peptide synthesis. I. The synthesis of a tetrapeptide. Journal of the American Chemical Society. 85, 2149-54. These peptide constructs can be adapted for various applications including half-life extension, drug delivery and tissue engineering. Additionally, isolated peptide constructs can be modified for conjugation to a drug molecule, prodrugs including a peptide therapeutic, a protein therapeutic, a small molecule drug, siRNA, mRNA, DNA, antibody, bispecific antibody, antigen binding fragment (Fab), a F(ab′)2, Fab′, single-chain variable fragment or scFv, di-scFv, single domain antibody sd-scFv hormone, enzyme, lipid, nanoparticles, microparticle, liposomes, lipidoids, exosomes, microvesicles to alter or modify their physical, chemical or biological behavior. Conjugation can occur directly, via a chemical entity, a cleavable or non-cleavable linker or through genetic fusion. Chemical modification of the isolated peptides described in the invention, can be achieved by solution or solid-phase peptide chemistry with standard tert-butoxycarbonyl (Boc) and 9-fluorenylmethoxycarbonyl (Fmoc) chemistry, however it can include other non-conventional linking strategies. A linker or a conjugation or crosslinking agent can be introduced into an isolated peptide by any known methods in the art. A chemical entity described herein can be any molecule, chemical functional group, material, or substrate that can be conjugated to the isolated amino acid construct by any known methods in the art.

The terminating groups and ionizable side chains of the isolated peptides may be ionized to form pharmaceutically acceptable salts. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The salt groups may be formed converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of include the non-toxic salts of the parent compound formed, for example, from non-toxic inorganic or organic acids. Alternatively, the peptides may form amphiphilic salts.

The term “peptide mimetic” as used herein refers to a molecule comprising any combination of natural amino acids, non-natural or non-standard amino acid, D-amino acids, β-alanine, γ-Aminobutyric acid (GABA), δ-Aminolevulinic acid, aminoisobutyric acid, dehydroalanine, and polyethylene glycol (PEG) or modifications thereof. The term also refers to molecules that have stability, conformational constraints, or pathways of assembly that are not possible with purely naturally occurring amino acid building blocks.

Accordingly, one aspect provided consists of an isolated peptide consisting essentially of chemically linked amino acid residues of the following sequence (X₁—X₂—X₃—X₄) wherein X₁ is isoleucine (Ile) or a conservative substitution thereof; X₂ is proline (Pro) or a conservative substitution thereof; X₃ is glycine (Gly) or a conservative substitution thereof; Xᵢᵥ is tyrosine (Tyr) or a conservative substitution thereof; The invention also describes an isolated peptide, wherein X₃ is independently selected from a bond, a non-coded, non-proteinogenic or non-standard amino acid linker including but not limited to β-alanine, γ-Aminobutyric acid (GABA), δ-Aminolevulinic acid, aminoisobutyric acid, dehydroalanine, PEG.

Accordingly, one embodiment of this invention includes an isolated peptide comprising chemically linked amino acid residues of the following sequence: X₁—X₂—X₃—X₄; where X₁ is an L- or D-amino acid; X₂ is proline, or a conservative substitution thereof; X₃ is selected from a group consisting of glycine or a conservative substitution thereof, a bond, and a non-coded, non-proteinogenic, or a non-standard amino acid linker; X₄ is an L- or D-amino acid; and said peptide is terminated with a biological entity.

A further embodiment of this invention includes the isolated peptide, where X₁ is isoleucine or a conservative substitution thereof.

Another further embodiment of this invention includes the isolated peptide, where said non-coded, non-proteinogenic, or non-standard amino acid linker is selected from the group consisting of β-alanine, γ-Aminobutyric acid (GABA), δ-Aminolevulinic acid, aminoisobutyric acid, dehydroalanine, and PEG.

Another further embodiment of this invention includes the isolated peptide, where said biological entity is selected from a group consisting of an amine, amide, imine, imide, azide, azo compound, carboxylic acid, carbonate, carboxylate, ester, alcohol, aldehyde, alkane, alkene, alkyne, halogens, ketone, acyl halide, boronic acid, boronic ester, borinic acid, borinic ester, hydroperoxide, peroxide, ether, hemiacetal, hemiketal, acetal, ketal, orthoester, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, fatty acid, myristic acid, palmytolyl, Fluorenylmethyloxycarbonyl or FMOC carbamate, Z (CBZ), Boc, tert-Butyl, cell surface receptor ligand, an antibody, bispecific antibody, an antibody-like molecule, Fab, Fc, or a portion thereof, an aptamer, a cytokine, hormone, a lectin, a lipid, nucleic acid, a carbohydrate, enzyme, biotin, avidin, streptavidin, steroid, protein A, protein G, a plasma albumin, a ligand, a therapeutic agent, fluorescent molecule, a binding molecule, a biodegradable non-amino acid polymer and any combinations thereof.

Another embodiment of this invention includes an isolated peptide comprising chemically linked amino acid residues of the following sequence: (X₅—X₆—X₇—X₈)ₙ; where X₅ is selected from one of the 21 naturally occurring amino acids; X₆ is proline, or a conservative substitution thereof; X₇ is selected from a group consisting of glycine or a conservative substitution thereof, a bond, and a non-coded, non-proteinogenic, or a non-standard amino acid linker; X₈ is selected from one of the 21 naturally occurring amino acids; N is an integer from 1-50, inclusive; and said peptide is terminated with a biological entity.

A further embodiment of this invention includes the isolated peptide, where X₅ is isoleucine or a conservative substitution thereof.

Another further embodiment of this invention includes the isolated peptide of claim 5, where the non-coded, non-proteinogenic, or non-standard amino acid linker is selected from the group consisting of β-alanine, γ-Aminobutyric acid (GABA), δ-Aminolevulinic acid, aminoisobutyric acid, dehydroalanine, and PEG.

Another further embodiment of this invention includes the isolated peptide, where the biological entity is selected from a group consisting of an amine, amide, imine, imide, azide, azo compound, carboxylic acid, carbonate, carboxylate, ester, alcohol, aldehyde, alkane, alkene, alkyne, halogens, ketone, acyl halide, boronic acid, boronic ester, borinic acid, borinic ester, hydroperoxide, peroxide, ether, hemiacetal, hemiketal, acetal, ketal, orthoester, cyanates, nitrate, nitrile, nitrite, nitro compound, nitroso compound, pyridine, thiol, sulfide, disulfide, sulfoxide, sulfone, sulfinic acid, sulfonic acid, thiocyanate, thione, thial, phosphine, phosphonic acid, phosphate, phosphodiester, fatty acid, myristic acid, palmytolyl, Fluorenylmethyloxycarbonyl or FMOC carbamate, Z (CBZ), Boc, tert-Butyl, cell surface receptor ligand, an antibody, bispecific antibody, an antibody-like molecule, Fab, Fc, or a portion thereof, an aptamer, a cytokine, hormone, a lectin, a lipid, nucleic acid, DNA, siRNA, mRNA, a carbohydrate, enzyme, biotin, avidin, streptavidin, steroid, protein A, protein G, a plasma albumin, a ligand, a therapeutic agent, fluorescent molecule, a binding molecule, a biodegradable non-amino acid polymer and any combinations thereof.

Another further embodiment of this invention includes the isolated peptide, where the amino acid sequence is selected from the group consisting of:

IPGY;
VPGY;
LPGY;
IPGF;
VPGF;
LPGF;
VPGW;
IPGW;
LPGW;
IPGY-VPGY-VPG;
IPGY-IPGY-IPG;
VPGY-VPGY-VPK;
IPGY-IPGY-IPK;
VPGY-VPGY-VPH;
IPGY-IPGY-IPH;
VPGY-VPGF-VPGY-V;
VPGY-VPGY-VPGY-V;
VPGY-VPGY-VPGY-L;
VPGY-VPGY-VPGY-VPGY-V;
VPGY-VPGY-VPGY-VPGY-VPG;
VPGY-VPGY-VPGY-VPGY-VPGY-VP;
VPGY-VPGY-VPGY-VPGY-VPGY-VPG;
VPGY-VPGY-VPGY-VPGY-VPGY-VPGY-VP;
VPGY-VPGY-VPGY-VPGY-VPGY-VPGY-VPGY;
VPGY-VPGY-VPGY-VPGY-VPGY-VPGY-VPGY-V;
VPGY-VPGY-VPGY-VPGY-VPGY-VPGY-VPGY-VPG;
VPGY-VPGY-VPGY-VPGY-VPGY-VPGY-VPGY-VPGY-V;
and
VPGY-VPGY-VPGY-VPGY-VPGY-VPGY-VPGY-VPGY-VP.

Another embodiment of this invention includes a half-life extension sequence comprising a single or plurality of any of the isolated peptides previously discussed.

A further embodiment of this invention includes the half-life extension sequence that also includes a therapeutic agent, drug molecule, or prodrug. Another further embodiment of this invention includes the half-life extension sequence that also includes a nanoparticle, microparticle, liposomes, lipidoids, exosomes, microvesicles, or any combination thereof. Another further embodiment of this invention includes the half-life extension sequence that also includes a quantum dot, a magnetic particle, gold nanoparticle, a silver nanoparticle, a carbon nanotube, a fullerene or any combination thereof.

Another further embodiment of this invention includes the half-life extension sequence where said therapeutic agent, drug molecule, or prodrug is selected from the group consisting of a peptide therapeutic, a protein therapeutic, a small molecule drug, siRNA, mRNA, DNA, an antibody, a bispecific antibody, a Fragment antigen binding (Fab), a F(ab′)2, Fab′, single-chain variable fragment or scFv, di-scFv, single domain antibody sd-scFv hormone, enzyme, and lipid, or any combination thereof.

Another embodiment of this invention includes a peptide aggregate comprising one or plurality of the isolated peptides previously discussed. Peptide aggregate may be formed spontaneously in aqueous media (e.g., water, a salt solution and/or a buffered solution) or serum, plasma, whole blood, or combination thereof. A further embodiment of this invention includes the aggregated peptide formed in aqueous, or chemical solvent (e.g., DMSO, isopropyl alcohol, acetone, ethanol, dioxane, acetonitrile, methanol, THF, or any combinations thereof), or a fraction of the dissolved peptides can be introduced (e.g., by injection, in-line mixing/mixer, dialysis) in an aqueous solvent, where the aggregate comprises a laminar structure, a solid structure, a porous structure or a combination thereof. Another further embodiment of this invention includes the aggregated peptide, where the aggregate has a size of 3 nm to 100 microns, or 5 nm to 100 microns.

Another embodiment of this invention includes a composition comprising at least one of the isolated peptides or peptide mimetic previously discussed, a peptide aggregate, or any combination thereof, where the composition is used as an excipient in a pharmaceutical product, or in a food product.

Another embodiment of this invention includes a composition comprising at least one of the isolated peptides or peptide mimetic previously discussed, a peptide aggregate, or any combination thereof, where the composition is a personal care composition including a lotion, cream, oils, gels or shampoo, ointment or any combination thereof.

Another embodiment of this invention includes a method of modulating surface and material property using any of the peptides or peptide mimetics previously discussed, where said peptide or peptide mimetic is chemically crosslinked to the tissue culture surface of a tissue culture dish by any known methods in the art, using Polyethylene, Polypropylene, Polystyrene, polyvinyl chloride, or borosilicate. For example, by crosslinking the peptides to the surface using a chemical crosslinking agent, electron beam exposure, gamma-radiation, UV irradiation. Crosslinking here can also be covalent, ionic, hydrophobic or others methods know in the art.

Another embodiment of this invention includes a method for modulating a least one or more of the following biological cell behavior: cell growth, viability, apoptosis, secretion, migration, or differentiation. This method includes contacting the biological cell with the cell of at least one of the peptides or peptide mimetics previously discussed, where the peptides constructs are cross-linked to the surface of the cells using a chemical crosslinking agent, ionic, hydrophobic or other methods know in the art. For example, the peptide can be crossed linked to the cells via an amine, thiol, or carboxyl functional group.

Another embodiment of this invention includes a method of modulating the release of an active drug, drug agent, or drug substance comprising any of the peptides and/or peptide mimetics previously discussed, wherein the active drug is covalently linked, encapsulated, or embedded using ionic interactions or hydrophobic interactions in the peptide aggregates by any know method in the art, and release of the drugs occurs by degradation of the peptide, or through the natural porosity of the aggregate. The porosity can be adjusted, based on a number of factors such as, but not limited to, desired release rates, molecular size and/or diffusion coefficient of the therapeutic agent or active agent.

Another embodiment of this invention includes a kit comprising at least one container containing an isolated peptide or peptide mimetic previously discussed.

In some embodiments, at least one terminus of the amino acid sequence can be modified and either the modified or unmodified peptides, peptide mimetic and or repeat units can be present in any form or shape, including but not limited to, an aggregate, vesicle, a particle, irregular or regular-shaped particles, a gel, or any combinations thereof. In some embodiments, peptide and peptide mimetic can be tuned to be stable over any period of time.

In some embodiments, at least one terminus of the amino acid sequence can be modified and either the modified or unmodified peptides, peptide mimetic and or repeat units can be present in any form or shape, including but not limited to, an aggregate, vesicle, a particle, irregular or regular-shaped particles, a gel, or any combinations thereof. In some embodiments, peptide and peptide mimetic can be tuned to be stable over any period of time.

When employed as pharmaceuticals, the isolated peptides or constructs can be administered as pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the isolated peptides or constructs in combination with one or more pharmaceutically acceptable carriers (excipients). In some embodiments, the composition is suitable for topical or intravenuous administration. In making the compositions of the invention, the active ingredient is typically mixed with an excipient, diluted by an excipient or enclosed within such a carrier in the form of, for example, a capsule, sachet, paper, or other container.

The amount of therapeutic agent or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. The pH of the preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the therapeutic agent can vary according to, for example, the particular use for which the treatment is made, the manner of administration of the compound, the health and condition of the patient, and the judgment of the prescribing physician. The proportion or concentration of the therapeutic agentin a pharmaceutical composition can vary depending upon a number of factors including dosage, chemical characteristics (e.g., hydrophobicity), and the route of administration. For example, the therapeutic agent can be provided in an aqueous physiological buffer solution containing about 0.1 to about 10% w/v of the therapeutic agent for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### EXAMPLES

### Example 1

### Design of Exemplary Aggregating Peptide Constructs Comprising 4-11 Amino Acids

It was observed that tropoelastin monomer secreted by elastogenic cells such as smooth muscle cells, endothelial cells, and fibroblasts can aggregate into organized spheres that ultimately gets incorporated into growing elastin fibers. Toonkool P, Regan D G, Kuchel P W, Morris M B, Weiss A S. J Biol Chem. 2001; 276:28042-28050 Wise S G, Mithieux S M, Raftery M J, Weiss A S. J Struct Biol. 2005; 149:273-281. A close analysis of the human tropoelastin sequence reveals a high abundance of the following amino acids: Glycine (Gly ˜28%), Alanine (Ala ˜21%), Valine (Val ˜14) Proline (Pro ˜12%) (see Table A below). Here, we designed libraries of isolated peptides, peptide mimetics and repeat units, comprising various combinations of certain amino acids. In particular, we discovered that certain key residues (e.g. Pro and Gly) that triggers clustering or controlled aggregation into well-defined nanostructures for application in tissue engineering and drug delivery.

TABLE A
Predicted Structural Class of Human Tropoelastin

| Molecular Weight | 68997.30 | m.w. |
|---|---|---|
| Length | 791 | |
| 1 microgram= | 14.493 | pMoles |
| Molar Extinction coefficient | 22600 ± 5% | |
| 1 A(280)= | 3.05 | mg/ml |

| Isoelectric Point | 10.43 | | |
|---|---|---|---|
| Charge at pH 7 | 39.15 | | |
| | Number | % by | % by |
| Amino Acid(s) | count | weight | frequency |
| Charged (RKHYCDE) | 74 | 14.9 | 9.36 |
| Acidic (DE) | 8 | 1.44 | 1.01 |
| Basic (KR) | 47 | 9.22 | 5.94 |
| Polar (NCQSTY) | 56 | 9.65 | 7.08 |
| Hydrophobic (AILFWV) | 355 | 46.71 | 44.88 |
| A Ala | 169 | 17.41 | 21.37 |
| C Cys | 2 | 0.3 | 0.25 |
| D Asp | 3 | 0.5 | 0.38 |
| E Glu | 5 | 0.94 | 0.63 |
| F Phe | 16 | 3.41 | 2.02 |
| G Gly | 223 | 18.44 | 28.19 |
| H His | 2 | 0.4 | 0.25 |
| I Ile | 17 | 2.79 | 2.15 |
| K Lys | 35 | 6.5 | 4.42 |
| L Leu | 55 | 9.02 | 6.95 |
| M Met | 1 | 0.19 | 0.13 |
| N Asn | 1 | 0.17 | 0.13 |
| P Pro | 99 | 13.93 | 12.52 |
| Q Gln | 10 | 1.86 | 1.26 |
| R Arg | 12 | 2.72 | 1.52 |
| S Ser | 16 | 2.02 | 2.02 |
| T Thr | 12 | 1.76 | 1.52 |
| V Val | 98 | 14.08 | 12.39 |
| W Trp | 0 | 0 | 0 |
| Y Tyr | 15 | 3.55 | 1.9 |

A library of highly scalable short linear peptides or their cyclic counterpart was designed that will aggregate in aqueous medium to form micro- and nano-aggregates (micro- and nano-particles) for use in drug delivery and tissue engineering. The aggregating peptides can be formulated as homogeneous (one group of unique aggregating peptide sequence) or heterogeneous (two or more groups of unique aggregating peptide sequences) mixtures and or in combination with small molecule drugs, DNA, siRNA, mRNA, excipients, stabilizers, protein, drugs for reducing drug toxicity, improving pharmacokinetics (PK), enhancing drug efficacy, targeting agents selectively to disease sites, delivering drugs to intracellular targets, and any combinations thereof. In accordance with some embodiments of one aspect described herein, a diverse library of aggregating peptides, e.g., 4-11, amino acids total in length, Table 1-3, was designed. The aggregating peptides can be formulated in aqueous media to generate a series of micro- and nano-structures (e.g., microparticles and nanoparticles) with a capability to control the size of the aggregates from nanometer to micrometer. These aggregating peptides can be used in various applications, e.g., for nanotherapeutics and nanomedicine, diagnostics, drug delivery, and tissue engineering applications.

For an experimental approach, a candidate peptide sequence can be synthesized as described herein, e.g., by solid-state peptide synthesis. The aggregating peptides sequences can also be produced as fusion peptides or protein by recombinant gene expression in bacteria (e.g. Escherichia coli) or mammalian cell lines such as the Chinese hamster ovary (CHO), HEK and COS cell lines or any methods known in the art used to produce protein. The aggregating peptide or fusion peptide construct can then be subjected to various formulation buffers and/or processing conditions to evaluate its aggregation potential. Characterization of any peptide aggregate, nano or nanostructures formed, e.g., size, shape, stability can be performed using any methods known in the art or as described in the Examples below.

Exemplary aggregating peptide comprising linear sequences (4-11 amino acid sequence) in three- and one-letter codes are shown in Tables 1-5. Each indicated amino acid sequence is designated with a number to which is referred throughout the specification. The short aggregating peptide sequences having the general formula:
a) X₁—X₂—X₃—X₄ (N-terminus to C-Terminus; N-C) or (C-terminus to N-Terminus; C-N)
b) X₁₁—X₁₂—X₁₁—X₁₀—X₉ (the bond between X₁₁ and X₁₂ is formed from the alpha-carboxyl group of X₁₁ and the alpha-amino group of X₁₂)
c) (X₅—X₆—X₇—X₈)ₙ (N-C and C-N)
d) random combinations of a, b, c;
   wherein X₁ is an L- or D-amino acid; X₂, X₆, and X₁₀ is proline, or a conservative substitution thereof; X₃, X₇, and X₁₁ is selected from a group consisting of glycine or a conservative substitution thereof, a bond, and a non-coded, non-proteinogenic, or a non-standard amino acid linker; X₄, X₈, and X₁₂ is an L- or D-amino acid; and said peptide is terminated with chemical group, molecule, peptide blocking group, peptide, or biological entity. The 4-11 amino acids constructs reported here were designed to test the ability to control aggregation properties and stability (Tables 1-3). Each peptide in the Tables 1-3 was prepared, for example, by FMOC-based solid-phase peptide synthesis and all of the peptide sequences were verified for >70% purity before by HPLC. The ability of these short hydrophobic peptide sequences to self-organize in aqueous media was then evaluated. As described in detail in the following Examples, the short peptides (as shown in Tables 1-3) formed a particulate suspension spontaneously within seconds in aqueous media and size of aggregate measured by Dynamic light scattering (DLS). These aggregating peptides can be mixed in various combinations and ratios (Table 3-5) to form particulate suspension of well-defined sized as measured by DLS.

Each of the aggregating peptide in Tables 1-4 was prepared by FMOC solid-phase peptide synthesis. For example, these peptides were synthesized on the acid sensitive Wang resin and cleaved from the resin with a solution mixture of trifluoroacetic acid/triisopropylsilane/water in a volume ratio of 9.5/2.5/2.5. Synthesized peptides were purified by reversed phase HPLC or flash column chromatography. The peptide sequences were purified by HPLC using a C18 5 μm 120 A 4.6*150 mm column in 0.1% TFA/H20 (buffer A) and 0.09% TFA in 80% ACN/20% H₂O (buffer B).

Tables 1-3 shows aggregating amino acid sequences (4-11 amino acid sequences) in three- and one-letter codes. Each indicated amino acid sequence is designated with an entry number to which is referred throughout the specification. Table 4 shows one-letter code of aggregating fusion sequence (for example peptide carrier fused to peptide drug) with G, GS and GGS linker. Each indicated amino acid sequence is designated with an entry number to which is referred throughout the specification. Table 5 shows one-letter code of combination of isolated aggregating peptide sequence combing different sequences. Each indicated amino acid sequence is designated with an entry number to which is referred throughout the specification.

TABLE 1
Design of aggregating peptide constructs
comprising 4 amino acids

| | General Formula: X₁-X₂-X₃-X₄ | One-letter |
|---|---|---|
| Entry | (N-C and C-N) | amino |
| # | Three-letter amino acids code | acids code |
| 01 | H-Val-Pro-Gly-Tyr-OH | VPGY |
| 02 | H-Val-Pro-Gly-Phe-OH | VPGF |
| 03 | H-Val-Pro-Gly-Ile-OH | VPGI |
| 05 | H-Val-Pro-Gly-His-OH | VPGH |
| 06 | H-Val-Pro-Gly-Trp-OH | VPGW |
| 07 | H-Lys-Pro-Gly-Tyr-OH | KPGY |
| 08 | H-Glu Pro-Gly-Tyr-OH | EPGY |
| 09 | H-Lys-Pro-Gly-Phe-OH | KPGF |
| 10 | H-Glu-Pro-Gly-Phe-OH | EPGF |
| 11 | H-Ile-Pro-Gly-Tyr-OH | IPGY |
| 12 | H-Thr-Pro-Gly-Tyr-OH | TPGY |
| 13 | H-Ile-Pro-Gly-Phe-OH | IPGF |
| 14 | H-Thr-Pro-Gly-Phe-OH | TPGF |

TABLE 2
Design of aggregating peptide constructs
comprising 5-11 amino acids

| | General Formula: | One-letter |
|---|---|---|
| Entry | X₁₁-X₁₂-X₁₁-X₁₀-X₉ | amino |
| # | (But not reversed sequence); | acids code |
| 28 | H-Gly-Tyr-Gly-Pro-Val-OH | GYGPV |
| 29 | H-Gly-Phe-Gly-Pro-Val-Gly- | GFGPVGYGPV |
| | Tyr-Gly-Pro-Val-OH | |

TABLE 3
Design of aggregating peptide constructs
comprising 8-11 amino acids

| | General Formula: (X₅-X₆-X₇-X₈)ₙ | One-letter |
|---|---|---|
| Entry | (N-C and C-N) | amino |
| # | and random combinations | acids code |
| 15 | H-Val-Pro-Gly-Tyr-Val-Pro-Gly- | VPGYVPGYVPG |
| | Tyr-Val-Pro-Gly-OH | |
| 16 | H-Ile-Pro-Gly-Tyr-Ile-Pro-Gly- | IPGYIPGYIPG |
| | Tyr-Ile-Pro-Gly-OH | |
| 17 | H-Val-Pro-Gly-Tyr-Val-Pro-Gly- | VPGYVPGYVPK |
| | Tyr-Val-Pro-Lys-OH | |
| 18 | H-Ile-Pro-Gly-Tyr-Ile-Pro-Gly- | IPGYIPGYIPK |
| | Tyr-Ile-Pro-Lys-OH | |
| 19 | H-Val-Pro-Gly-Tyr-Val-Pro-Gly- | VPGYVPGYVPH |
| | Tyr-Val-Pro-His-OH | |
| 20 | H-Ile-Pro-Gly-Tyr-Ile-Pro-Gly- | IPGYIPGYIPH |
| | Tyr-Ile-Pro-His-OH | |
| 21 | H-Pro-Val-Gly-Tyr-Val-Pro-Gly- | PVGYVPGF |
| | Phe-OH | |
| 22 | H-Val-Pro-Gly-Tyr-Pro-Val-Gly- | VPGYPVGF |
| | Phe-OH | |
| 23 | H-Pro-Val-Gly-Tyr-Pro-Val-Gly- | PVGYPVGF |
| | Phe-OH | |
| 24 | H-Pro-Val-Gly-Tyr-Val-Pro-Phe- | PVGYVPFG |
| | Gly-OH | |
| 25 | H-Val-Phe-Pro-Gly-Tyr-Pro-Val- | VFPGYPVG |
| | Gly-OH | |
| 26 | H-Gly-Pro-Val-Gly-Tyr-Val-Gly- | GPVGYVGPFG |
| | Pro-Phe-Gly-OH | |
| 27 | H-Tyr-Gly-Val-Gly-Phe-Val-Gly- | YGVGFVGPGP |
| | Pro-Gly-Pro-OH | |
| 30 | H-Tyr-Gly-Pro-Val-Tyr-Gly-Pro- | YGPVYGPV |
| | Val-OH | |

TABLE 4
Aggregating peptide fusion sequence (i.e peptide
covalently linked to therapeutic drug) with G,
GS and GGGS linker.

| Entry # | Peptide drug fusion with linker or spacer |
|---|---|
| 31 | VPGY-CHHHRHSF |
| 32 | VPGY-G-CHHHRHSF |
| 33 | VPGY-GS-CHHHRHSF |
| 34 | VPGY-GGGS-CHHHRHSF |

TABLE 5
Mixed aggregating peptide constructs mixed with
peptide drug fusion in ratios but not
limited to 1:1

| Entry # | Mixed sequences (1:1 or 1:1:1) |
|---|---|
| 35 | VPGY:KPGY |
| 36 | VPGY:EPGY |
| 37 | VPGY:TPGY |
| 38 | VPGY:KPGY:TPGY |
| 39 | VPGY:VPGY-GGGS-CHHHRHSF |
| 40 | KPGY:VPGY-GGGS-CHHHRHSF |
| 41 | TPGY:VPGY-GGGS-CHHHRHSF |
| 42 | VPGY:VPGY-CHHHRHSF |
| 43 | VPGY:VPGY-G-CHHHRHSF |
| 44 | VPGY:VPGY-GS-CHHHRHSF |
| 45 | VPGY:VPGY-GGGS-CHHHRHSF |

### Example 2

### Dynamic Light Scattering Studies

Described below are DLS studies that showed the amino acid sequences in Tables 1-4 each prepared at a concentration of about 5 mg/mL to 50 mg/mL in buffer and the resulting nanoaggregates formed as measured by DLS. Micro- and Nano-aggregation of these peptide constructs (Table 1-5) is induced by directly mixing, precipitation, or dialysis in aqueous buffer. The peptide concentration of the resulting mixture or suspensions are about 5 mg/mL to about 50 mg/mL (weight/volume, w/v). Nano and Micro aggregation can also be induced by adding a precipitation while stirring at about 100 rpm to about 500 rpm for about 1-20 mins. The stirring speed can be varied to control size homogeneity. Dynamic light scattering (DLS) analysis of these constructs confirmed nanostructures formation as described below.

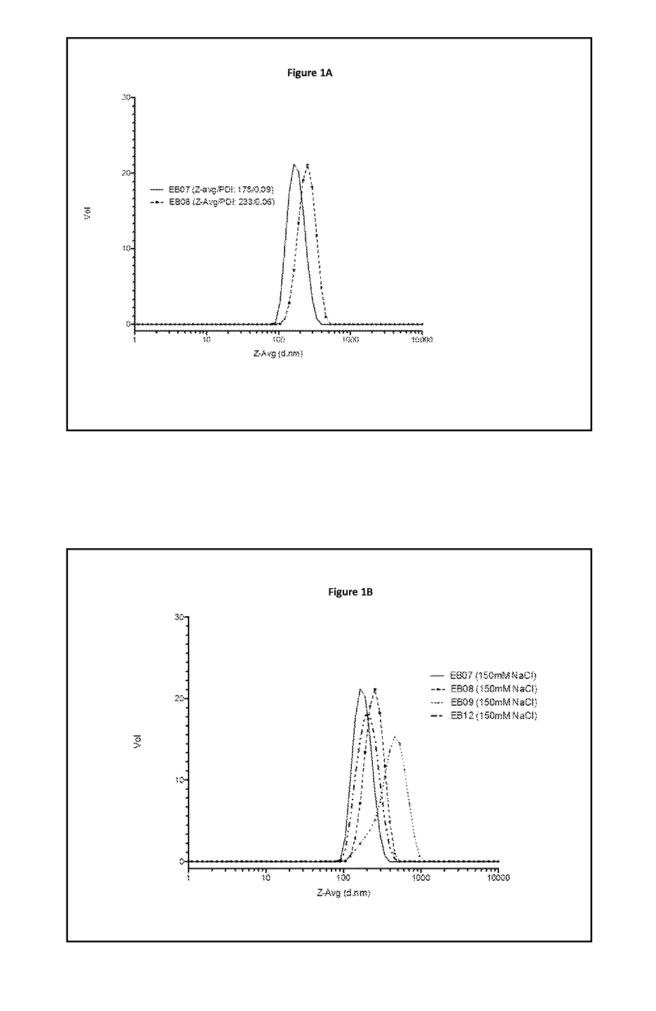

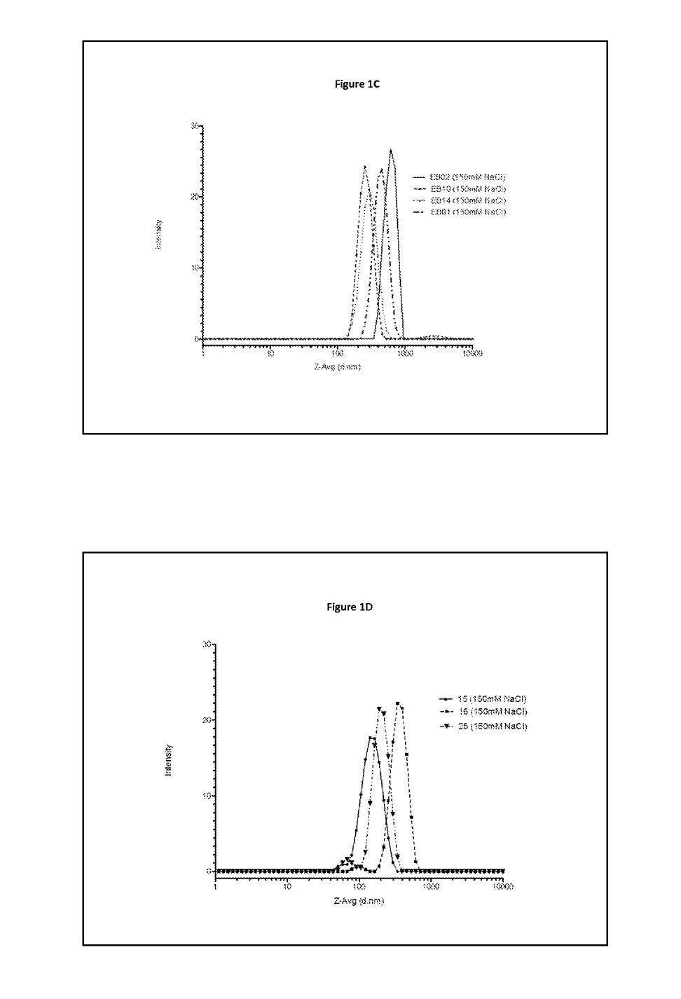

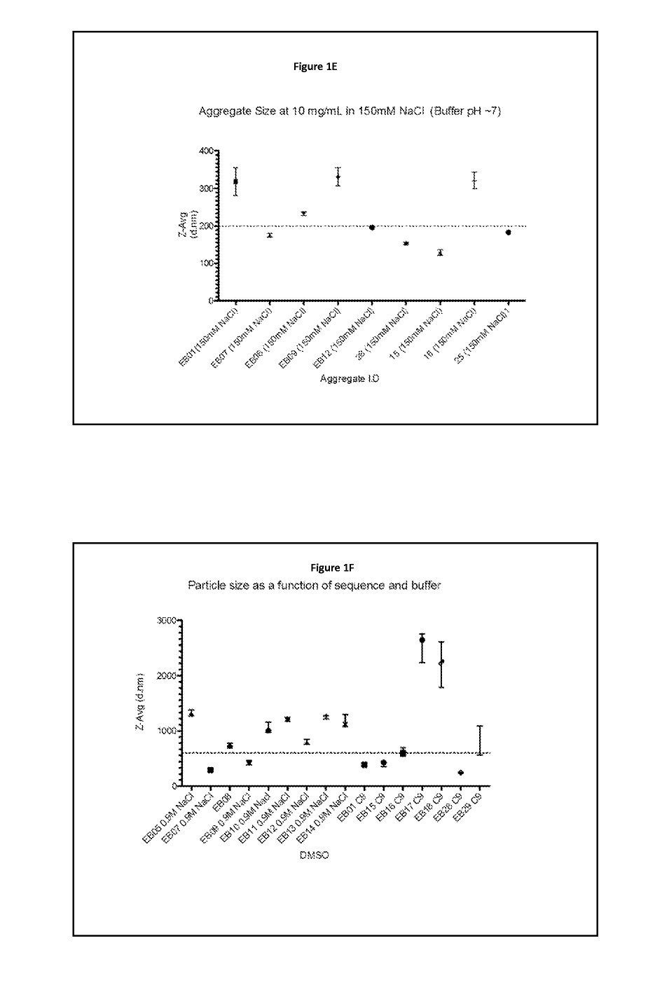

FIGS. 1-4 shows DLS size distribution plot of representative peptide aggregates as a function of peptide sequence in various buffer, concentration and pH (ranging from pH3 to pH9).

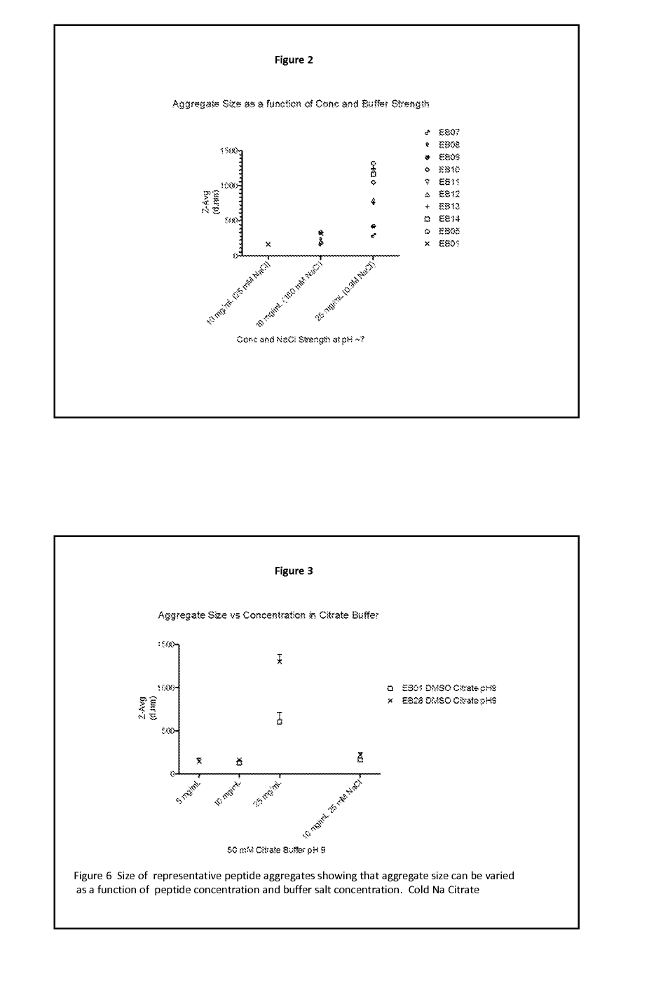

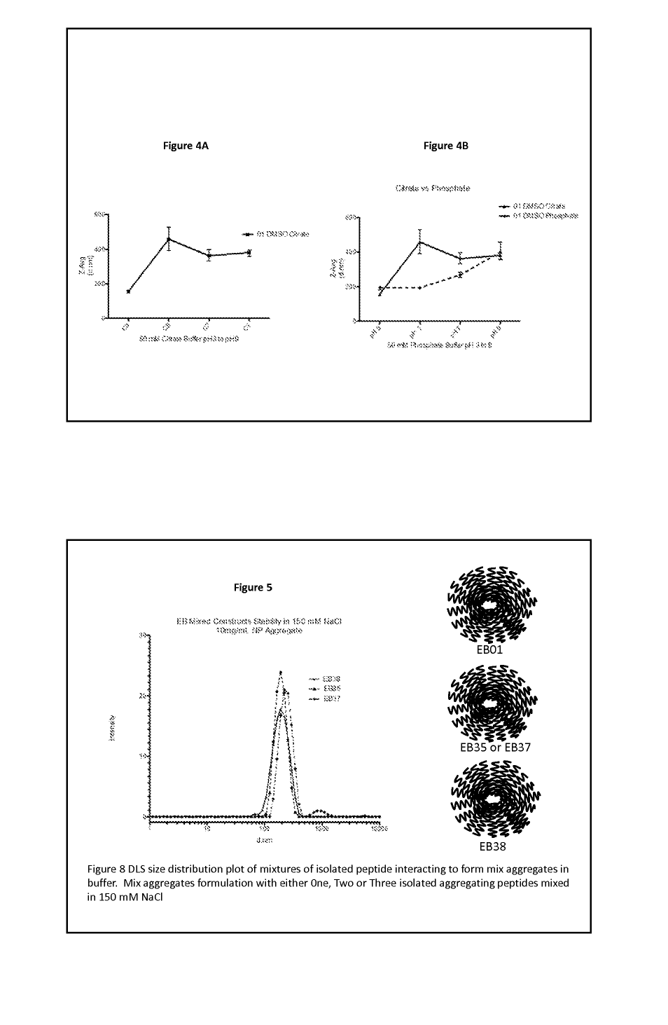

A wide range of peptide-based particulates (nanoparticles and or aggregates) can be generated from the peptide library (Table 1-5) as a function of peptide sequence, peptide concentration, buffer strength, and buffer pH). In these studies, aggregating peptide constructs were dissolved in an organic solvent (e.g., but not limited to, DMSO, Isopropyl alcohol, ethanol, acetone, dioxane, acetonitrile, methanol, and THF) at 300-800 mg/ml and a fix volumes was then injected or mixed with cold acid, neutral or alkaline buffer solution (e.g., but not limited to, 150 mM NaCl, Sodium Citrate, Acetate, Phosphate) while stirring or mixing. The cold precipitation method (e.g., using a cold saline medium) can more efficiently induce peptide aggregation that are near monodisperse or with very low polydispersities (from 0.05 to 0.8). Different concentrations and/or types of salts could be used to prepare the buffer solution, depending on the solubility of the peptide constructs in the respective solution. In some embodiments, any buffer solution such as PBS, acetate, succinate and citrate buffer could be used instead. The resulting particles size varied with peptide concentration from about 5 mg/ml to about 80 mg/ml with low polydispersities. The ability to generate a wide range of particles sizes with low polydispersities can be desirable or advantageous in certain applications, e.g., but not limited to, nanotechnology and/or drug delivery. In some embodiments, the peptides described herein can form nanoparticles having a particle size with low polydispersity (e.g., with a polydispersity index of less than 0.5, less than 0.4, less than 0.3, less than 0.2, less than 0.1, or lower). In other embodiments, the peptides described herein can form nanoparticles having a particle size with a polydispersity index of about 0.5 or higher, e.g., at least about 0.5, at least about 0.6, at least about 0.7 or higher. The aggregating peptide sequences can form aggregates of various size and stability based on formulation strategy. FIGS. 4A-5B shows that the size and stability of the peptide aggregates can be controlled when formulated in buffer as a function of pH.

FIG. 5 shows DLS size distribution plot of mixtures of isolated peptide interacting to form mix aggregates in buffer. Mix aggregates formulation with either one, two or three isolated aggregating peptides mixed in 150 mM NaCl. Each unique aggregating peptide constructs were dissolved in an organic solvent (e.g., but not limited to, DMSO, Isopropyl alcohol, ethanol, acetone, dioxane, acetonitrile, methanol, and THF) in separate vials at 300-800 mg/ml and a fix volumes of each was combined then injected or mixed with cold acid, neutral or alkaline buffer solution (e.g., but not limited to, 150 mM NaCl, sodium citrate, acetate, and phosphate) while stirring or mixing.

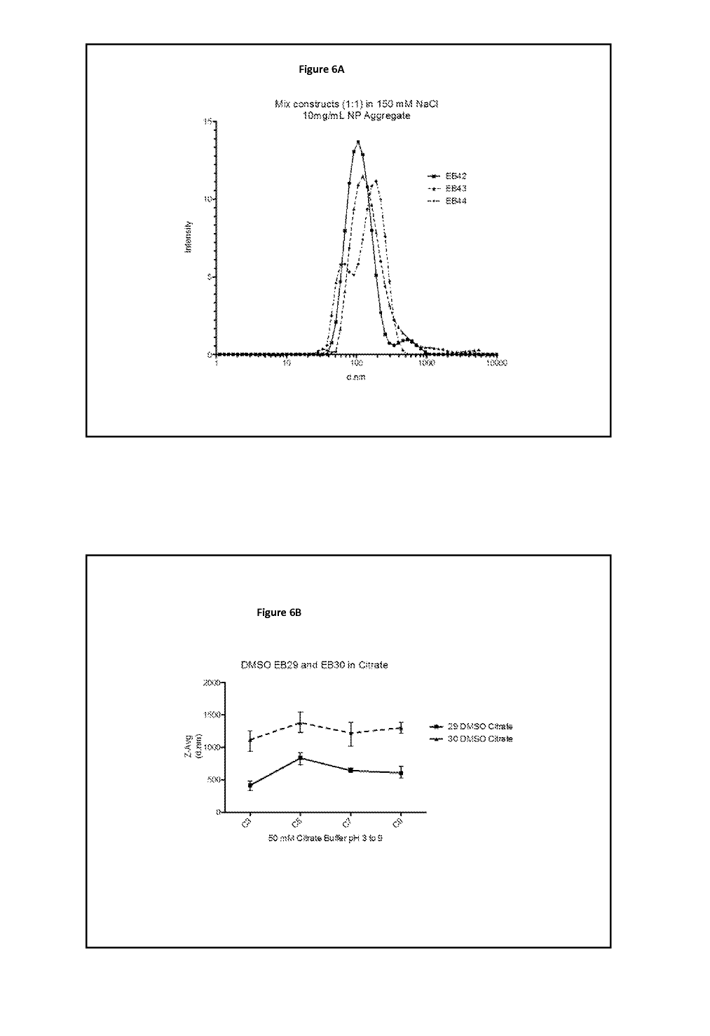

FIG. 6 shows DLS size distribution plot of mixtures of isolated peptide and peptide drug fusion interacting to form mix aggregates in buffer. Mix aggregates formulated (1:1) for a final concentration of 10 mg/mL in 150 mM NaCl. Each unique aggregating peptide constructs were dissolved in an organic solvent that is suitable for the specific sequence (e.g., but not limited to, DMSO, Isopropyl alcohol, ethanol, acetone, dioxane, acetonitrile, methanol, and THF) in separate vials at 300-800 mg/ml and a fix volumes of each was combined then injected or mixed with cold acid, neutral or alkaline buffer solution (e.g., but not limited to, 150 mM NaCl, Sodium Citrate, Acetate, Phosphate) while stirring or mixing.

All references cited herein are incorporated by reference herein in their entireties.

1-65. (canceled)

66. An aggregated peptide construct, wherein the aggregated construct is a mixture of peptides having the amino acid sequence VPGI or VPGY, wherein the sequence is N-terminus to C-Terminus (N-C) or C-terminus to N-Terminus (C-N).

67. The construct of claim 66, wherein the peptides have the amino acid sequence VPGI, wherein the sequence is N-terminus to C-Terminus (N-C).

68. The construct of claim 66, wherein the peptides have the amino acid sequence VPGY, wherein the sequence is N-terminus to C-Terminus (N-C).

69. The construct of claim 66, wherein the peptides are terminated with a biological entity.

70. The construct of claim 69, wherein the biological entity is an amine, amide, imine, imide, azide, azo compound, carboxylic acid, carbonate, carboxylate, ester, alcohol, aldehyde, alkane, alkene, alkyne, halogens, ketone, acyl halide, boronic acid, boronic ester, borinic acid, borinic ester, hydroperoxide, peroxide, ether, hemiacetal, hemiketal, acetal, ketal, orthoester, cyanates, nitrate, nitrile, nitrite, nitro compound, nitroso compound, pyridine, thiol, sulfide, disulfide, sulfoxide, sulfone, sulfuric acid, sulfonic acid, thiocyanate, thione, thial, phosphine, phosphonic acid, phosphate, phosphodiester, fatty acid, myristic acid, palmytolyl, Fluorenylmethyloxycarbonyl or FMOC carbamate, Z (CBZ), Boc, tert-Butyl, cell surface receptor ligand, an antibody, bispecific antibody, an antibody-like molecule, Fab, Fc, or a portion thereof, an aptamer, a cytokine, hormone, a lectin, a lipid, nucleic acid, a carbohydrate, enzyme, biotin, avidin, streptavidin, steroid, protein A, protein G, a plasma albumin, a ligand, a therapeutic agent, fluorescent molecule, a binding molecule, a biodegradable non-amino acid polymer, DNA, siRNA, and mRNA.

71. The construct of claim 66, wherein the peptides are terminated with a therapeutic agent.

72. The construct of claim 66, wherein the peptides have a terminal covalently linked therapeutic agent, wherein said therapeutic agent is covalently linked to the peptide via a linker linker is selected from GS, (GS)ₘ GGGS, and (GGGS)ₘ, wherein m is 1-25.

73. The construct of claim 66, wherein the aggregate comprises a laminar structure, a solid structure, a porous structure, and combinations thereof.

74. The construct of claim 66, wherein the aggregated construct forms a nanoparticle or a microparticle.

75. The construct of claim 66, wherein the aggregated construct has a size of 3 nm to 100 microns.

76. The construct of claim 66, wherein the aggregated construct has a size of 5 nm to 100 microns.

77. The construct of claim 66, wherein the peptides have the amino acid sequence VPGI, wherein the sequence is C-terminus to N-Terminus (C-N).

78. The construct of claim 66, wherein the peptides have the amino acid sequence VPGY, wherein the sequence is C-terminus to N-Terminus (C-N).

79. An isolated peptide having a four amino acid sequence of VPGI, wherein the sequence is N-terminus to C-Terminus (N-C) or C-terminus to N-Terminus (C-N).

80. The isolated peptide of claim 79, wherein the sequence is N-terminus to C-Terminus (N-C).

81. The isolated peptide of claim 79, wherein the sequence is C-terminus to N-Terminus (C-N).


SEQUENCE LISTING (74 sequences)

SEQ ID NO: 1 - protein, 4 aa, Artificial Sequence
  Description of Artificial Sequence Synthetic peptide
XXXX

SEQ ID NO: 2 - protein, 4 aa, Artificial Sequence
  Description of Artificial Sequence Synthetic peptide
XXXX

SEQ ID NO: 3 - protein, 8 aa, Artificial Sequence
  Description of Artificial Sequence Synthetic peptide
CHHHRHSF

SEQ ID NO: 4 - protein, 4 aa, Artificial Sequence
  Description of Artificial Sequence Synthetic peptide
VPGY

SEQ ID NO: 5 - protein, 4 aa, Artificial Sequence
  Description of Artificial Sequence Synthetic peptide
VPGF

SEQ ID NO: 6 - protein, 4 aa, Artificial Sequence
  Description of Artificial Sequence Synthetic peptide
VPGI

SEQ ID NO: 7 - protein, 4 aa, Artificial Sequence
  Description of Artificial Sequence Synthetic peptide
VPGH

SEQ ID NO: 8 - protein, 4 aa, Artificial Sequence
  Description of Artificial Sequence Synthetic peptide
VPGW

SEQ ID NO: 9 - protein, 4 aa, Artificial Sequence
  Description of Artificial Sequence Synthetic peptide
KPGY

SEQ ID NO: 10 - protein, 4 aa, Artificial Sequence
  Description of Artificial Sequence Synthetic peptide
EPGY

SEQ ID NO: 11 - protein, 4 aa, Artificial Sequence
  Description of Artificial Sequence Synthetic peptide
KPGF

SEQ ID NO: 12 - protein, 4 aa, Artificial Sequence
  Description of Artificial Sequence Synthetic peptide
EPGF

SEQ ID NO: 13 - protein, 4 aa, Artificial Sequence
  Description of Artificial Sequence Synthetic peptide
IPGY

SEQ ID NO: 14 - protein, 4 aa, Artificial Sequence
  Description of Artificial Sequence Synthetic peptide
TPGY

SEQ ID NO: 15 - protein, 4 aa, Artificial Sequence
  Description of Artificial Sequence Synthetic peptide
IPGF

SEQ ID NO: 16 - protein, 4 aa, Artificial Sequence
  Description of Artificial Sequence Synthetic peptide
TPGF

SEQ ID NO: 17 - protein, 12 aa, Artificial Sequence
  Description of Artificial Sequence Synthetic peptide
VPGYCHHHRHSF

SEQ ID NO: 18 - protein, 13 aa, Artificial Sequence
  Description of Artificial Sequence Synthetic peptide
VPGYGCHHHRHSF

SEQ ID NO: 19 - protein, 14 aa, Artificial Sequence
  Description of Artificial Sequence Synthetic peptide
VPGYGSCHHHRHSF

SEQ ID NO: 20 - protein, 16 aa, Artificial Sequence
  Description of Artificial Sequence Synthetic peptide
VPGYGGGSCHHHRHSF

SEQ ID NO: 21 - protein, 200 aa, Artificial Sequence
  Description of Artificial Sequence Synthetic polypeptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 22 - protein, 200 aa, Artificial Sequence; identical to SEQ ID NO: 21
  Description of Artificial Sequence Synthetic polypeptide

SEQ ID NO: 23 - protein, 200 aa, Artificial Sequence; identical to SEQ ID NO: 21
  Description of Artificial Sequence Synthetic polypeptide

SEQ ID NO: 24 - protein, 54 aa, Artificial Sequence
  Description of Artificial Sequence Synthetic polypeptide
GSGSGSGSGSGSGSGSGSGSGSGSGSGSGSGSGSGSGSGSGSGSGSGSGSGGGS

SEQ ID NO: 25 - protein, 100 aa, Artificial Sequence
  Description of Artificial Sequence Synthetic polypeptide
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 26 - protein, 11 aa, Artificial Sequence
  Description of Artificial Sequence Synthetic peptide
VPGYVPGYVPG

SEQ ID NO: 27 - protein, 11 aa, Artificial Sequence
  Description of Artificial Sequence Synthetic peptide
IPGYIPGYIPG

SEQ ID NO: 28 - protein, 11 aa, Artificial Sequence
  Description of Artificial Sequence Synthetic peptide
VPGYVPGYVPK

SEQ ID NO: 29 - protein, 11 aa, Artificial Sequence
  Description of Artificial Sequence Synthetic peptide
IPGYIPGYIPK

SEQ ID NO: 30 - protein, 11 aa, Artificial Sequence
  Description of Artificial Sequence Synthetic peptide
VPGYVPGYVPH

SEQ ID NO: 31 - protein, 11 aa, Artificial Sequence
  Description of Artificial Sequence Synthetic peptide
IPGYIPGYIPH

SEQ ID NO: 32 - protein, 8 aa, Artificial Sequence
  Description of Artificial Sequence Synthetic peptide
PVGYVPGF

SEQ ID NO: 33 - protein, 8 aa, Artificial Sequence
  Description of Artificial Sequence Synthetic peptide
VPGYPVGF

SEQ ID NO: 34 - protein, 8 aa, Artificial Sequence
  Description of Artificial Sequence Synthetic peptide
VFPGYPVG

SEQ ID NO: 35 - protein, 10 aa, Artificial Sequence
  Description of Artificial Sequence Synthetic peptide
GPVGYVGPFG

SEQ ID NO: 36 - protein, 10 aa, Artificial Sequence
  Description of Artificial Sequence Synthetic peptide
YGVGFVGPGP

SEQ ID NO: 37 - protein, 8 aa, Artificial Sequence
  Description of Artificial Sequence Synthetic peptide
YGPVYGPV

SEQ ID NO: 38 - protein, 5 aa, Artificial Sequence
  Description of Artificial Sequence Synthetic peptide
XXXXX

SEQ ID NO: 39 - protein, 250 aa, Artificial Sequence
  Description of Artificial Sequence Synthetic polypeptide
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 40 - protein, 250 aa, Artificial Sequence; identical to SEQ ID NO: 39
  Description of Artificial Sequence Synthetic polypeptide

SEQ ID NO: 41 - protein, 5 aa, Artificial Sequence
  Description of Artificial Sequence Synthetic peptide
GYGPV

SEQ ID NO: 42 - protein, 10 aa, Artificial Sequence
  Description of Artificial Sequence Synthetic peptide
GFGPVGYGPV

SEQ ID NO: 43 - protein, 4 aa, Artificial Sequence
  Description of Artificial Sequence Synthetic peptide
LPGY

SEQ ID NO: 44 - protein, 4 aa, Artificial Sequence
  Description of Artificial Sequence Synthetic peptide
LPGF

SEQ ID NO: 45 - protein, 4 aa, Artificial Sequence
  Description of Artificial Sequence Synthetic peptide
IPGW

SEQ ID NO: 46 - protein, 4 aa, Artificial Sequence
  Description of Artificial Sequence Synthetic peptide
LPGW

SEQ ID NO: 47 - protein, 11 aa, Artificial Sequence
  Description of Artificial Sequence Synthetic peptide
IPGYVPGYVPG

SEQ ID NO: 48 - protein, 13 aa, Artificial Sequence
  Description of Artificial Sequence Synthetic peptide
VPGYVPGFVPGYV

SEQ ID NO: 49 - protein, 13 aa, Artificial Sequence
  Description of Artificial Sequence Synthetic peptide
VPGYVPGYVPGYV

SEQ ID NO: 50 - protein, 13 aa, Artificial Sequence
  Description of Artificial Sequence Synthetic peptide
VPGYVPGYVPGYL

SEQ ID NO: 51 - protein, 17 aa, Artificial Sequence
  Description of Artificial Sequence Synthetic peptide
VPGYVPGYVPGYVPGYV

SEQ ID NO: 52 - protein, 19 aa, Artificial Sequence
  Description of Artificial Sequence Synthetic peptide
VPGYVPGYVPGYVPGYVPG

SEQ ID NO: 53 - protein, 22 aa, Artificial Sequence
  Description of Artificial Sequence Synthetic peptide
VPGYVPGYVPGYVPGYVPGYVP

SEQ ID NO: 54 - protein, 23 aa, Artificial Sequence
  Description of Artificial Sequence Synthetic peptide
VPGYVPGYVPGYVPGYVPGYVPG

SEQ ID NO: 55 - protein, 26 aa, Artificial Sequence
  Description of Artificial Sequence Synthetic peptide
VPGYVPGYVPGYVPGYVPGYVPGYVP

SEQ ID NO: 56 - protein, 28 aa, Artificial Sequence
  Description of Artificial Sequence Synthetic peptide
VPGYVPGYVPGYVPGYVPGYVPGYVPGY

SEQ ID NO: 57 - protein, 29 aa, Artificial Sequence
  Description of Artificial Sequence Synthetic peptide
VPGYVPGYVPGYVPGYVPGYVPGYVPGYV

SEQ ID NO: 58 - protein, 31 aa, Artificial Sequence
  Description of Artificial Sequence Synthetic polypeptide
VPGYVPGYVPGYVPGYVPGYVPGYVPGYVPG

SEQ ID NO: 59 - protein, 33 aa, Artificial Sequence
  Description of Artificial Sequence Synthetic polypeptide
VPGYVPGYVPGYVPGYVPGYVPGYVPGYVPGYV

SEQ ID NO: 60 - protein, 34 aa, Artificial Sequence
  Description of Artificial Sequence Synthetic polypeptide
VPGYVPGYVPGYVPGYVPGYVPGYVPGYVPGYVP

SEQ ID NO: 61 - protein, 8 aa, Artificial Sequence
  Description of Artificial Sequence Synthetic peptide
VPGYKPGY

SEQ ID NO: 62 - protein, 8 aa, Artificial Sequence
  Description of Artificial Sequence Synthetic peptide
VPGYEPGY

SEQ ID NO: 63 - protein, 8 aa, Artificial Sequence
  Description of Artificial Sequence Synthetic peptide
VPGYTPGY

SEQ ID NO: 64 - protein, 12 aa, Artificial Sequence
  Description of Artificial Sequence Synthetic peptide
VPGYKPGYTPGY

SEQ ID NO: 65 - protein, 20 aa, Artificial Sequence
  Description of Artificial Sequence Synthetic peptide
VPGYVPGYGGGSCHHHRHSF

SEQ ID NO: 66 - protein, 20 aa, Artificial Sequence
  Description of Artificial Sequence Synthetic peptide
KPGYVPGYGGGSCHHHRHSF

SEQ ID NO: 67 - protein, 20 aa, Artificial Sequence
  Description of Artificial Sequence Synthetic peptide
TPGYVPGYGGGSCHHHRHSF

SEQ ID NO: 68 - protein, 16 aa, Artificial Sequence
  Description of Artificial Sequence Synthetic peptide
VPGYVPGYCHHHRHSF

SEQ ID NO: 69 - protein, 17 aa, Artificial Sequence
  Description of Artificial Sequence Synthetic peptide
VPGYVPGYGCHHHRHSF

SEQ ID NO: 70 - protein, 18 aa, Artificial Sequence
  Description of Artificial Sequence Synthetic peptide
VPGYVPGYGSCHHHRHSF

SEQ ID NO: 71 - protein, 20 aa, Artificial Sequence
  Description of Artificial Sequence Synthetic peptide
VPGYVPGYGGGSCHHHRHSF

SEQ ID NO: 72 - protein, 8 aa, Artificial Sequence
  Description of Artificial Sequence Synthetic peptide
PVGYPVGF

SEQ ID NO: 73 - protein, 8 aa, Artificial Sequence
  Description of Artificial Sequence Synthetic peptide
PVGYVPFG

SEQ ID NO: 74 - protein, 4 aa, Artificial Sequence
  Description of Artificial Sequence Synthetic peptide
GGGS